(19) United States
(12) Patent Application Publication
(10) Pub. No.: US 2003/0104028 A1
(43) Pub. Date: Jun. 5, 2003

(54) Rate limiting barriers for implantable devices and methods for fabrication thereof

(76) Inventors: Syed F.A. Hossainy, Fremont, CA (US); Wouter E. Roorda, Palo Alto, CA (US); Eugene T. Michal, San Francisco, CA (US)

Correspondence Address: Squire, Sanders & Dempsey L.L.P., Suite 300, One Maritime Plaza, San Francisco, CA, 94111, US

(21) Appl. No.: 09/998,083

(22) Filed: Nov. 29, 2001

(51) Int. Cl. A61F 2/00

(52) U.S. Cl. 424/424; 427/2.25

(57) ABSTRACT

A coating for a medical device, particularly for a drug eluting stent, is described. The coating comprises a layer of an organic polymer component containing a therapeutic substance and a layer of an inorganic component for controlling the rate of release of the substance. The inorganic component according to embodiments of the invention includes gold or diamond-like carbon.

### BACKGROUND OF THE INVENTION

1. Field of the Invention

This invention relates to the field of medical devices, especially devices used for delivery of drugs. Particularly, this invention is directed to coatings for drug delivery devices, such as drug eluting vascular stents. More particularly, this invention is directed to coatings for controlling the rate of release of drugs from stents and methods of fabricating the same.

2. Description of Related Art

In the field of medical technology, there is frequently a necessity to administer drugs locally. To provide an efficacious concentration to the treatment site, systemic administration of such medication often produces adverse or toxic side effect for the patient. Local delivery is a preferred method of treatment in that smaller total levels of medication are administered in comparison to systemic dosages, but are concentrated at a specific site.

In the treatment of vascular disorders, such as arteriosclerosis, intracoronary stents are now a standard adjunct to balloon angioplasty. Stenting is now preferred to balloon angioplasty in that it eliminates vasospasm, tacks dissections to the vessel wall, and reduces negative remodeling.

Stents can be made from interconnected struts that are usually between 50 and 150 microns wide. Being made of a metal (for instance, stainless steel), bare stents have to be modified so as to provide means for allowing the strut to deliver a drug. Accordingly, stents are being modified by forming a polymer coating, containing a drug, on the surface of the stent.

Currently, a typical embodiment of a coating used to achieve local drug delivery via stent comprises a three-layer composition, as shown by FIG. 1 and described subsequently. The three layer composition includes a drug-polymer layer 3 serving as a reservoir for the drug, an optional primer polymer layer 2 for improving adhesion of the drug-polymer layer 3 to the surface of the stent 1, and an optional topcoat polymer layer 4 for reducing the rate of release of the drug. The medicine to be administered will have a sustained release profile from the drug-polymer layer 3 through the topcoat polymer layer 4.

To the extent that the mechanical functionality of stents has been optimized in recent years, it has been determined that continued improvements could be done by means of pharmacological therapies. For the purposes of pharmacological therapy, it is important to maintain the concentration of the drug at a therapeutically effective level for an acceptable period of time. Hence, controlling a rate of release of the drug from the stent is important, especially in such a way so as to decrease the release rate of the drug from the underlying matrix.

In addition, existing stents have low radio-opacity and are often not well discernable under X-ray imaging. It is preferred for stents to present a bright image to allow a physician the ability to discern the stent at the desired location with more precision. This beneficial property can be achieved if the radio-opacity of the stent is enhanced. Therefore, increased radio-opacity is an additional desired quality.

In view of the foregoing, coatings for reducing the rate of release a therapeutic substance from implantable devices, such as stents, are desired. The coatings should prolong the residence time of the drug in the patient and provide for an increase in the radio-opacity of the device.

### SUMMARY

According to one aspect of this invention, a coating for a medical device is disclosed, the coating comprising a layer of an organic polymer component containing a therapeutic substance and a layer of an inorganic component for reducing the rate of release of the therapeutic substance.

According to another aspect of this invention, a method for fabricating a medical device is described, the method comprising forming a coating on the device, the coating comprising an organic polymer component containing a therapeutic substance and an inorganic component for reducing the rate of release of the substance.

According to one embodiment of the invention, the inorganic component providing for the reduction of the rate of release of the therapeutic substance includes gold or diamond-like carbon.

According to another embodiment of the invention, the gold surface of the coating can be modified with a passivating agent, such as an adduct of poly(ethylene glycol) with a thiol, a derivative of a hyaluronic acid, a derivative of heparin, or a combination thereof.

### BRIEF DESCRIPTION OF THE DRAWINGS

The features and advantages of the embodiments of the present invention will become better understood with regard to the following description, appended claims, and accompanying drawings where:

FIG. 1 schematically depicts a cross-section of a known and currently used multi-layered polymeric coating for stents.

FIG. 2 schematically depicts a cross-section of an embodiment of a coating on a stent according to the present invention.

### DETAILED DESCRIPTION

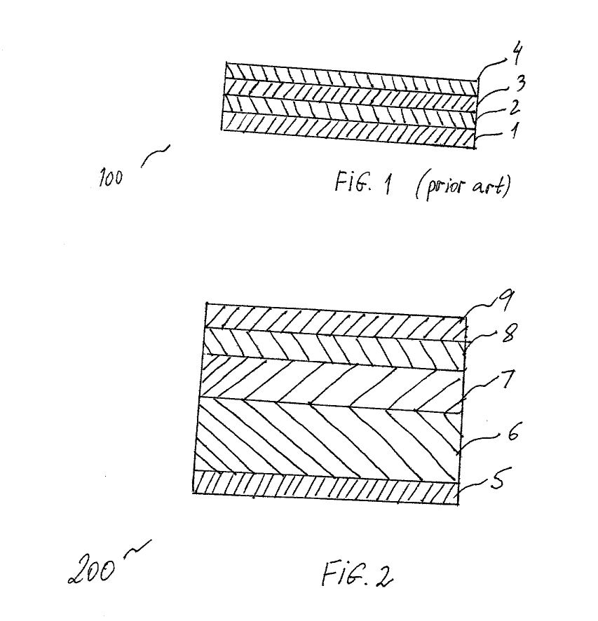

FIG. 1 shows a cross-section of a medical device 100 having a polymer coating. This coating is currently known and used on medical devices, particularly on stents. According to this embodiment, a stent 1 is coated with a primer polymer layer 2 and by a drug-polymer layer 3. The drug-polymer layer 3 comprises a polymer binder and a drug, dispersed in the binder, to be administered via the stent 1. Finally, a polymer topcoat layer 4 is applied on top of the drug-polymer layer 3 for reducing the rate of release of the drug.

FIG. 2 shows an embodiment 200 of the coated stent according to the present invention. This embodiment comprises a stent 5, an optional primer layer 6, a drug-polymer layer 7, and an optional topcoat layer 8. A layer of inorganic compound 9 is applied onto the topcoat layer 8, or directly onto the drug-polymer layer 7 if the topcoat layer 8 is not used.

Examples of inorganic compounds used to form layer 9 include gold and diamond-like carbon (DLC), also known to those having ordinary skill in the art as tetrahedral amorphous carbon. The term “diamond-like carbon” is commonly used because an amorphous carbon can be produced in which a proportion of the carbon atoms are bonded similar to that of diamond and the structure of which resembles diamond in many ways. DLC is a hard but flexible, chemically inert and atomically dense material. Accordingly, DLC is wear, corrosion and diffusion resistant as well as biocompatible.

The gold or DLC layer 9 substantially reduces the rate of release of the biologically active agent from the drug-polymer layer 7. In addition to the rate controlling effect, the gold or DLC layer 9 also substantially increases the radio-opacity of the stent.

Optionally, in order to further modify of the rate of release, pores can be created in the layer 9 by using any suitable technique, such as laser drilling. If desired, the layer 9 can be optionally coated with another polymer layer.

In case of the gold-containing coatings, as for instance described in Examples 1 and 2 below, it is also desirable to improve their long term in vivo response and to reduce the possibility of inflammation, platelet activation and fibrin deposition. In order to improve the biocompatibility of gold layer 9, the gold surface is modified by a passivating agent.

The modification of the gold surface can be achieved by the reaction of gold with thiol containing compounds (sometimes referred to as mercapto compounds). Several biocompatible agents are modified with thiol-containing ligands. These agents include poly(ethylene glycol) (PEG), hyaluronic acid, heparin or a heparin derivative containing a hydrophobic counter-ion, as shown in the Examples 4-6 below. It should be understood that any combination of thiolated PEG, hyaluronic acid, heparin or a heparin derivative containing a hydrophobic counter-ion can also be used for modification of the gold surface. The thiolated agents are used to covalently bind to the gold surface, thus improving the gold's in vivo response.

The coating of the present invention has been described in conjunction with a stent. However, the coating can also be used with a variety of other medical devices. Examples of the implantable medical device, that can be used in conjunction with the embodiments of this invention include stent-grafts, grafts (e.g., aortic grafts), artificial heart valves, cerebrospinal fluid shunts, pacemaker electrodes, axius coronary shunts and endocardial leads (e.g., FINELINE and ENDOTAK, available from Guidant Corporation). The underlying structure of the device can be of virtually any design. The device can be made of a metallic material or an alloy such as, but not limited to, cobalt-chromium alloys (e.g., ELGILOY), stainless steel (316L), “MP35N,” “MP20N,” ELASTINITE (Nitinol), tantalum, tantalum-based alloys, nickel-titanium alloy, platinum, platinum-based alloys such as, e.g., platinum-iridium alloy, iridium, gold, magnesium, titanium, titanium-based alloys, zirconium-based alloys, or combinations thereof. Devices made from bioabsorbable or biostable polymers can also be used with the embodiments of the present invention.

“MP35N” and “MP20N” are trade names for alloys of cobalt, nickel, chromium and molybdenum available from Standard Press Steel Co. of Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum.

A copolymer of ethylene and vinyl alcohol (EVAL) is one example of a polymer used to fabricate the drug-polymer layer 7, the optional primer layer 6 and/or the optional topcoat layer 8. EVAL has the general formula —[CH₂—CH₂]ₘ—[CH₂—CH(OH)]ₙ—. EVAL is a product of hydrolysis of ethylene-vinyl acetate copolymers and may also be a terpolymer including up to 5 molar % of units derived from styrene, propylene and other suitable unsaturated monomers.

A brand of copolymer of ethylene and vinyl alcohol distributed commercially under the trade name EVAL by Aldrich Chemical Co. of Milwaukee, Wis., and manufactured by EVAL Company of America of Lisle, Illinois, can be used.

Other suitable polymers can also be used to form a drug-polymer layer 7, the optional primer layer 6, and/or the optional topcoat layer 8. Representative examples include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane; poly(amino acids), cyanoacrylates, poly(trimethylene carbonate), poly(iminocarbonate), co-poly(ether-esters) (e.g. PEO/PLA), polyalkylene oxalates, polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers, vinyl halide polymers and copolymers (such as polyvinyl chloride), polyvinyl ethers (such as polyvinyl methyl ether), polyvinylidene halides (such as polyvinylidene fluoride and polyvinylidene chloride), polyacrylonitrile, polyvinyl ketones, polyvinyl aromatics (such as polystyrene), polyvinyl esters (such as polyvinyl acetate), copolymers of vinyl monomers with each other and olefins (such as ethylene-methyl methacrylate copolymers, acrylonitrile-styrene copolymers, ABS resins, and ethylene-vinyl acetate copolymers), polyamides (such as Nylon 66 and polycaprolactam), alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. On top of drug-polymer layer 8, a topcoat layer (not shown) can be optionally applied.

The polymer can be applied to the stent by dissolving the polymer in a solvent and applying the resulting composition on the stent or immersing the stent in the composition. Representative examples of some suitable solvents include N,N-dimethylacetamide (DMAC) having the formula CH₃—CO—N(CH₃)₂, N,N-dimethylformamide (DMFA) having the formula H—CO—N(CH₃)₂, tethrahydrofurane (THF) having the formula C₄H₈O, dimethylsulphoxide (DMSO) having the formula (CH₃)₂C═O, or trifluoro acetic anhydride (TFAA) having the formula (CF₃—CO)₂O.

There are no limitations on the drugs to be included within the drug-polymer layer 7. For example, the active agent of the drug could be designed to inhibit the activity of vascular smooth muscle cells. It can be directed at inhibiting abnormal or inappropriate migration and/or proliferation of smooth muscle cells to inhibit restenosis.

Generally speaking, the active agent of the drug can include any substance capable of exerting a therapeutic or prophylactic effect in the practice of the present invention. The drug may include small molecule drugs, peptides, proteins, oligonucleotides, or double-stranded DNA.

Examples of the drugs which are usable include antiproliferative substances such as actinomycin D, or derivatives and analogs thereof. Synonyms of actinomycin D include dactinomycin, actinomycin IV, actinomycin I₁, actinomycin X₁, and actinomycin C₁.

The active agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel, docetaxel, methotrexate, azathioprine, vincristine, vinblastine, fluorouracil, doxorubicin hydrochloride, and mitomycin.

Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin.

Examples of such cytostatic or antiproliferative agents include angiopeptin, angiotensin converting enzyme inhibitors such as captopril, cilazapril or lisinopril, calcium channel blockers (such as nifedipine), colchicine, fibroblast growth factor (FGF) antagonists, fish oil (ω-3-fatty acid), histamine antagonists, lovastatin (an inhibitor of HMG-CoA reductase, a cholesterol lowering drug), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide.

An example of an antiallergic agent is permirolast potassium. Other therapeutic substances or agents which may be appropriate include alpha-interferon, genetically engineered epithelial cells, rapamycin and dexamethasone.

### EXAMPLES

Embodiments of the present invention are illustrated by the following Examples.

### Example 1

A composition is prepared by mixing the following components:

(a) between about 0.1 mass % and about 15 mass %, for example, about 2.0 mass % of EVAL;

(b) between about 0.05 mass % and about 1.0 mass %, for example, about 0.7 mass % of actinomycin D (AcD); and

(c) the balance, DMAC solvent.

The composition is applied onto the stent, and dried. A primer (e.g., the above formulation without the therapeutically active compound) can be optionally applied on the surface of the bare stent.

For a stent having a length of 13 mm and diameter of 3 mm, the total amount of solids of the drug-polymer layer is about 100 micrograms (corresponding to the thickness of between about 5 and 6 microns). “Solids” means the amount of the dry residue deposited on the stent after all volatile organic compounds (e.g., the solvent) have been removed.

A composition comprising between about 0.1 mass % and about 15 mass %, for example, about 2.0 mass % of EVAL and the balance of DMAC, is applied onto the dried drug-polymer layer and dried, to form the optional topcoat. The topcoat can have, for example, a total solids weight of about 500 μg.

Following the formation of the topcoat layer, a layer of gold is applied onto the topcoat layer by any method known to those having ordinary skill in the art, such as for example, by sputtering, plasma deposition or spraying a gold suspension in EVAL.

### Example 2

A composition can be prepared by mixing the following components:

a) between about 0.1 mass % and about 15 mass %, for example, about 2.0 mass % of EVAL; p1 (b) between about 0.05 mass % and about 1.0 mass %, for example, about 0.7 mass % of b-estradiol; and

(c) the balance, DMAC solvent.

The composition is applied onto a stent as described in Example 1, to form a drug-polymer layer with about 200 μg of total solids. A composition comprising between about 0.1 mass % and about 15 mass %, for example, about 2.0 mass % of EVAL and the balance of DMAC is applied onto the dried drug-polymer layer, to form the optional topcoat layer having has a total solids weight of about 200 μg. Followed the formation of the topcoat layer, a layer of gold is applied onto the topcoat layer by any conventional method mentioned in Example 1.

### Example 3

A composition can be prepared by mixing the following components:

(a) between about 0.1 mass % and about 15 mass %, for example, about 2.0 mass % of EVAL;

(b) between about 0.05 mass % and about 1.0 mass %, for example, about 0.7 mass % of b-estradiol; and

(c) the balance, DMAC solvent.

The composition is applied onto a stent to form a drug-polymer layer with about 300 μg of total solids. A composition coating comprising between about 0.1 mass % and about 15 mass %, for example, about 2.0 mass % of EVAL and the balance of DMAC is applied onto the dried drug-polymer layer to form an optional topcoat layer having a total solids weight of about 300 μg.

Following the formation of the topcoat layer, a layer of diamond-like carbon (DLC), an inorganic additive, is applied onto the topcoat layer by any method known to those having ordinary skill in the art, for example, by chemical vapor deposition (CVD), ion-beam assisted deposition (IBAD), or molecular beam epitaxy (MBE).

The three examples of the formulations above can be summarized as shown in Table 1.

TABLE 1
A Summary of the Formulations of Examples 1-3

| | Polymer | Drug in | Solids in | Polymer in | Solids in | |
|---|---|---|---|---|---|---|
| | in drug- | drug- | dry drug- | the | the dry | |
| | polymer | polymer | polymer | topcoat | topcoat | Inorganic |
| Example | layer | layer, | layer, μg | layer | layer, μg | additive |
| 1 | EVAL, 2% | AcD, 0.7% | 100 | EVAL, 2% | 500 | Gold |
| 2 | EVAL, 2% | b-estradiol, | 200 | EVAL, 2% | 200 | Gold |
| | | 0.7% | | | | |
| 3 | EVAL, 2% | b-estradiol, | 300 | EVAL, 2% | 300 | DLC |
| | | 0.7% | | | | |

### Example 4

The gold coated stent described in Examples 1 or 2 is passivated with a passivating agent. A thiol-modified PEG (PEG-thiol) manufactured by Shearwater Corp. of Huntsville, Ala., is used as the passivating agent. In particular, methoxylated PEG-thiol is used representing PEG terminated with thiol on one end and with the methoxy group on the other, having a general formula OCH₃—[CH₂—CH₂—O—CH₂—CH₂]ₙ—SH, with a molecular weight of about 5,000 Daltons.

The gold coated stent described in Examples 1 or 2 above is immersed into a solution of PEG-thiol for a period of between about 1 hour and about 24 hours. During this period of time the PEG-thiol bonds to the gold surface via covalent bonding. The concentration of the PEG-thiol solution is between about 0.1 and about 5 g/l.

### Example 5

Hyaluronic acid, which is a linear polysaccharide composed of disaccharide units of N-acetylglucosamine and D-glucoronic acid, is used. In hyaluronic acid, uronic acid and the aminosugar are linked by alternating β-1,4 and β-1,3 glucosidic bonds.

Hyaluronic acid is coupled to cystamine, NH₂CH₂CH₂—S—S—CH₂CH₂NH₂, in the presence of 1-ethyl-3(3-dimethylaminopropyl)carbodiimide, having the formula CH₃—CH₂—N═C═N—CH₂—CH₂—CH₂—N(CH₃)₂, also known as carbodiimide or EDC. Hyaluronic acid reacts with EDC first and forms an O-acylisourea, an amine-reactive intermediate. This intermediate is unstable in aqueous environment and immediately reacts with cystamine utilizing cystamine's amino groups. This reaction is maintained for about 4 hours, in a neutral or slightly acidic medium with a pH of abut 5 to 7.

The dilsulfide linkage of the product of the coupling of hyaluronic acid to cystamine is then reduced using one of the appropriate reducing agents. Examples of such reducing agents include sodium cyanoborohydride, having the formula NaBH₃CN; or 1,4-dimercapto-2,3-butanediol (also known as dithiothreitol or the Cleland's reagent), having the formula HS—CH₂—CH(OH)—CH(OH)—CH₂—SH (DTT), or tris-(2-carboxyethyl)phosphine (TCEP), having the formula (P—CH₂—CH₂—COOH)₃.

As a result of the reaction of reduction, free mercapto groups —SH are generated. Since the mercapto groups are prone to oxidation, the final modifying solution containing these groups is stored in an inert atmosphere (e.g., under argon or nitrogen).

The gold coated stent described in Examples 1 or 2 is immersed into the thiolated hyaluronic acid-based modifying solution of PEG-thiol for a period of between about 1 hour and about 24 hours. The concentration of the solution is between about 0.1 and about 5 g/l.

### Example 6

The same procedure is used as in Example 5, except instead of hyaluronic acid, heparin is thiolated via its carboxyl groups. The reaction of thiolation is the same as in Example 5, including the coupling of heparin to cystamine followed by generating mercapto groups by a reaction of reduction using the same reducing agents.

The gold coated stent described in Examples 1 or 2 is immersed into the thiolated heparin-derived modifying solution of PEG-thiol for a period of between about 1 hour and about 24 hours. The concentration of the solution is between about 0.1 and about 5 g/l.

Having described the invention in connection with several embodiments thereof, modification will now suggest itself to those having ordinary skill in the art. As such, the invention is not to be limited to the described embodiments

What is claimed is:

1. A coating for a medical device, said coating comprising:
(a) a layer of an organic polymer component containing a therapeutic substance; and
(b) a layer of an inorganic component for reducing the rate of release of said therapeutic substance.

2. The coating of claim 1, wherein said medical device is a stent.

3. The coating of claim 1, wherein said organic polymer component is fabricated of a copolymer of ethylene and vinyl alcohol.

4. The coating of claim 1, wherein said therapeutic substance comprises actinomycin D, estradiol, paclitaxel, docetaxel, or rapamycin.

5. The coating of claim 1, wherein said layer of the inorganic component comprises gold.

6. The coating of claim 5, wherein said layer of the inorganic component is modified with a passivating agent.

7. The coating of claim 6, wherein said passivating agent is selected from a group consisting of an adduct of poly(ethylene glycol) with a thiol, a derivative of a hyaluronic acid, heparin, a derivative of heparin containing hydrophobic counter-ions, and a combination thereof.

8. The coating of claim 5, wherein said layer of the inorganic component contains pores.

9. The coating of claim 1, wherein said layer of the inorganic component is formed on said layer of the organic polymer component.

10. The coating of claim 1, further including a polymeric coating disposed on said layer of the organic polymer component, wherein said layer of the inorganic component is formed on the polymeric coating.

11. The coating of claim 1, wherein said layer of the inorganic component is fabricated of diamond-like carbon.

12. A method for fabricating a coating for a medical device, the method comprising forming a coating on said device, said coating comprising a layer of an organic polymer component containing a therapeutic substance and a layer of an inorganic component for reducing the rate of release of said substance.

13. The method of claim 12, wherein said medical device is a stent.

14. The method of claim 12, wherein said layer of the organic polymer component is fabricated of a copolymer of ethylene and vinyl alcohol.

15. The method of claim 12, wherein said therapeutic substance comprises actinomycin D, estradiol, paclitaxel, docetaxel, or rapamycin.

16. The method of claim 12, wherein said layer of inorganic component includes gold.

17. The method of claim 16, wherein said layer of inorganic component is deposited by sputtering, plasma deposition or spraying a suspension of gold in a polymeric material.

18. The method of claim 12, wherein said layer of the inorganic component contains pores.

19. The method of claim 12, further comprising modifying said layer of inorganic component with a passivating agent.

20. The method of claim 19, wherein said passivating agent is selected from a group consisting of an adduct of poly(ethylene glycol) with a thiol, a derivative of a hyaluronic acid, a derivative of heparin, and a combination thereof.

21. The method of claim 11, wherein said layer of the inorganic component includes diamond-like carbon.

22. The method of claim 21, wherein said diamond-like carbon is deposited by chemical vapor deposition, ion beam assisted deposition, or molecular beam epithaxy.
